(19) United States
(12) Patent Application Publication
(10) Pub. No.: US 2005/0221473 A1
(43) Pub. Date: Oct. 6, 2005

(54) Sensor array integrated circuits

(75) Inventors: Valery M. Dubin, Portland, OR (US); Ken David, Beaverton, OR (US); Andrew A. Berlin, San Jose, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Intel Corporation, Santa Clara, CA (US)

(21) Appl. No.: 10/814,982

(22) Filed: Mar. 30, 2004

(51) Int. Cl. C12M 1/34; C12Q 1/68

(52) U.S. Cl. 435/287.2; 435/288.7; 435/6

(57) ABSTRACT

An apparatus includes a condensed array addressed device; and a spectroscope optically coupled to the condensed array addressed device. A method includes determining bonding and/or lack-of-bonding of a target molecule to a condensed array addressed device by characterizing a subsequent rate of electrolysis on the condensed array addressed device. A method includes fabricating a condensed array addressed device using damascene patterning.

### BACKGROUND OF THE INVENTION

1. Field Of The Invention

Embodiments of the invention relate generally to the field of biological and/or chemical sensing. More particularly, embodiments of the invention relate to integrating electronic memory with sensing devices, processes for sensing with integrated memory sensing devices and processes for making integrated memory sensing devices.

2. Background Information

Electrical impedance spectroscopy is well known in the literature. An Intel-funded project at the University of California at Berkeley has demonstrated that two electrodes separated by a gap can be used to detect hybridization of DNA through impedance change. However, this demonstration was done in a discrete system using an off-the-shelf impedance monitoring instrument.

Meanwhile, Nanogen corporation has demonstrated selectively enhanced capture and concentration of reagents using applied electric fields to enhance motion of analytes towards affinity reagents located on top of an SRAM. The analytes are then detected using optical fluorescence.

Heretofore, no sensor array integrated circuits exist which contain many different sensors to perform diagnosis and hazardous chemical analysis. Further, no sensor array integrated circuits exist that are co-integrated with electronic circuits to perform data storage, data analysis and/or data transfer/receiving. What is needed is a solution that simultaneously meets these needs.

### BRIEF DESCRIPTION OF THE DRAWINGS

The drawings accompanying and forming part of this specification are included to depict certain aspects of embodiments of the invention. A clearer conception of the embodiments of the invention, and of the components and operation of systems provided with embodiments of the invention, will become more readily apparent by referring to the exemplary, and therefore nonlimiting, embodiments illustrated in the drawings, wherein identical reference numerals designate the same elements. The embodiments of the invention may be better understood by reference to one or more of these drawings in combination with the description presented herein. It should be noted that the features illustrated in the drawings are not necessarily drawn to scale.

FIG. 1 illustrates a block schematic representation of a hand held device including a sensor array, representing an embodiment of the invention.

FIG. 2A illustrates a schematic top view of a sensor array, representing an embodiment of the invention.

FIG. 2B illustrates a schematic cross sectional view of a sensor array, representing an embodiment of the invention.

FIG. 3A illustrates a schematic top view of a sensor array, representing an embodiment of the invention.

FIG. 3B illustrates a schematic cross sectional view of a sensor array, representing an embodiment of the invention.

### DETAILED DESCRIPTION OF THE INVENTION

The embodiments of the invention and the various features and advantageous details thereof are explained more fully with reference to the nonlimiting embodiments that are illustrated in the accompanying drawings and detailed in the following description. Descriptions of well known starting materials, processing techniques, components and equipment are omitted so as not to unnecessarily obscure the embodiments of the invention in detail. It should be understood, however, that the detailed description and the specific examples, while indicating preferred embodiments of the invention, are given by way of illustration only and not by way of limitation. Various substitutions, modifications, additions and/or rearrangements within the spirit and/or scope of the underlying inventive concept will become apparent to those skilled in the art from this disclosure.

Embodiments of the invention can solve the problem of scaling and integration to reduce cost and enhance reliability. Currently, sensors are fabricated by using discrete technology which increases cost and reduces reliability of the sensors.

Embodiments of the invention can also solve the problem of response time. Sending samples to lab for analysis sometime takes weeks. Hand held devices containing thousands, much less millions, of sensors are not currently available to perform analysis at the point of care.

Embodiments of the invention can also solve the problem of rapidly accessing and reading information regarding sample reactivity to a large series of specific functional groups and their combinations. Sensors to analyze a sample with regard to a series of groups are not currently available in an array addressed format.

Embodiments of the invention can also solve the problem of selective reading and storage of the information contained on chemical species. Sensor arrays that immobilize chemicals to be analyzed are not currently available.

An embodiment of the invention can comprise a machine including a condensed array addressed device; and a spectroscope optically coupled to the condensed array addressed device. An embodiment of the invention can include a process of sensing a molecule using this machine. An embodiment of the invention can comprises a process including determining bonding and/or lack-of-bonding of a target molecule to a condensed array addressed device by characterizing a subsequent rate of electrolysis on the condensed array addressed device. An embodiment of the invention can comprise a data structure including results obtained using this process. An embodiment of the invention can comprise a process including fabricating a condensed array addressed device using damascene patterning. An embodiment of the invention can include a condensed array addressed device produced by this process.

These, and other, aspects of embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of embodiments of the invention without departing from the spirit thereof, and embodiments of the invention include all such substitutions, modifications, additions and/or rearrangements.

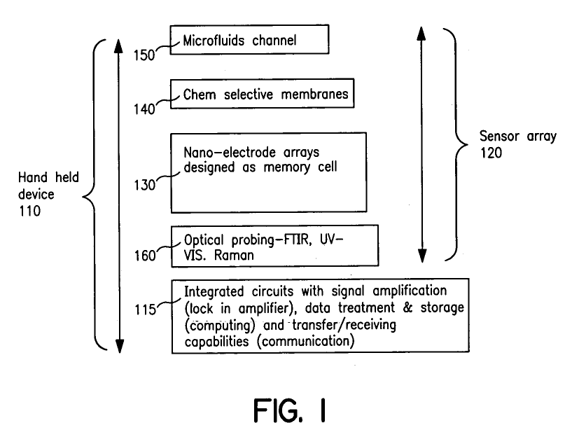

Referring to FIG. 1, a schematic block diagram of an embodiment of the invention is depicted in which a hand held device 110 includes a sensor array 120. Of course, the hand held device 110 (or other embodiments of the invention) can include a plurality of sensor arrays.

The hand held device 110 can include additional integrated circuits 115 with signal amplification (e.g., lock in amplifier), data treatment and storage (computing) and transfer/receiving capabilities (communications). For example, the hand held device can include logic (e.g., ASIC), memory (e.g., cache, buffers, FLASH, WORM, cards, drives, etc.), video display, power circuits (e.g., for addressing, activating, time domain integrating, refreshing, etc.) and/or signal processing circuits (e.g., amplifiers, (de)modulators, filters, antenna, etc.). The hand held device 110 can be coupled mechanically, electronically, optically and/or informationally to additional components (not shown in FIG. 1) to define a system.

The sensor array 120 can be integrated mechanically, electronically, optically and/or informationally to the balance of the hand held device 110. The sensor array 120 can be in-the-field (e.g., hot) swappable to expedite rapid, repetitive sampling, thereby facilitating on-the-spot data collection from a large number of samples and/or over a long duration.

The sensor array 120 can include a condensed array address device. Of course, the sensor array 120 can include a plurality of condensed array address devices. The condensed array address device can include a nano-electrode array 130. Of course, condensed array address device can include a plurality of nano-electrode arrays. The nano-electrode array 130 can be designed (configured) as a memory cell in a 1d (row or column), 2d (array) or even 3d (matrix) arrangement. The condensed array address device can be integrated mechanically, electronically and/or optically with the balance of the sensor array. For instance, the nano-electrode array 130 can include a 2d array of individually addressable single-wall carbon nanotube electrode cells (each cell having two or more functional electrodes) electronically integrated with the balance of the sensor array 120.

The sensor array 120 can include one or more selective membranes. The selective membranes can include chemically selective membrane 140 and/or a biologically selective membrane. The selective membranes can include polymer, ceramic and/or metal structures having one or more 1d, 2d, or 3d (interconnected and/or mutually exclusive) porous networks. The porous networks can be defined by apertures, holes, tubes, conduits, funnels and/or other shapes and the surfaces of these porous networks can have portions that are hydrophilic, hydrophobic, acidic, basic, wetted by surfactant, etc.) The selective membranes can be integrated mechanically, electronically and/or optically with the balance of the sensor array 120.

The sensor array 120 can include a microfluidic(s) channel 150. A primary function of the microfludic(s) channel is to maneuver an analyte to an appropriate position relative to electrodes on the sensor array 120. The microfluidic channel 150 can include one or more feeds, reservoirs, digesters, manifolds, pumps, mixing chambers, venturis, jets, valves, sumps and/or drains. The microfluidic(s) channel 150 can be integrated mechanically, electronically and/or optically with the balance of the sensor array 120.

The sensor array 120 can include characterization instrumentation. The characterization instrumentation can include optical probing instrumentation 160. The optical probing instrumentation 160 can include infra-red (e.g., Fourier transform infrared (FTIR)), ultraviolet-visible and/or Raman spectroscopy components. The spectroscopy components can include source(s), filter(s), polarizer(s), mirrors, beam splitters-combiners, apertures and detectors. The characterization instrumentation can be integrated mechanically, electronically and/or optically with the balance of the sensor array 120. Spectroscopy can be modulated to improve sensitivity (increase signal to noise ratios) by using electromodulation on nano-electrodes (i.e. modulated AC potential applied to nano-electrodes or photomodulation by using shopper on the way of the light beam. Electro-modulation on selected electrode will allow probe selected electrodes and bio-species binded on the surface of these electrodes.

An embodiment of the invention can include a sensor array integrated circuit containing nano-electrode arrays configured as a high density memory cell array which is capable to write information from biological and/or chemical molecules by immobilizing (adsorption) chemical species on biased electrode(s) as well as accessing and reading the information corresponding to specific chemical functional groups contained between two nano-electrodes by measuring current between these electrodes using applied alternative (pulse) voltages. As noted above, additional capabilities can be integrated such as microfluidics channels, chemically selective membrane, DC charge sensing, and total internal IR. Wireless communication and computing capabilities can be also integrated to perform storage, treatment/processing and transfer/receiving data/information (for example high density memory arrays of nano-electrodes can be built on top of standard CMOS/bi-polar chips).

An embodiment of the invention can include a sensor array containing an array of nano-electrodes having a cell size corresponding to repeated spacing between electrodes from approximately 5 nm to approximately 200 μm, preferably from approximately 5 nm to approximately 1000 nm. This pitch dimension can be reduced to approximately 0.8 nm size, or even smaller if the electrode(s) is(are) a single wall carbon nano-tube or a carbon nano-fiber. The sensor arrays can be designed as memory cell arrays which are capable of writing information from biological and/or chemical molecules by immobilizing (adsorption) chemical species on the biased electrodes as well as then accessing and reading, for example, a single bit of information corresponding to specific chemical functional group contained between the two electrodes by measuring, for example, current (impedance vs frequency) between these electrodes using applied alternative (pulse) voltages. Reading the information can be repeated (accumulated) (time domain expanded) to increase single to noise ratios.

Embodiments of the invention can include additional capabilities, optionally integrated, such as micro-fluidic(s) channel(s), chemical selective membrane(s) with the same or different pore sizes of from approximately 5 μm to approximately 1000 nm to sort molecules by their sizes. An embodiment of the invention can include characterization capabilities, optionally integrated, such as total internal optical paths for light (e.g., IR) to determine and/or validate chemical functional group(s) adsorbed on the electrodes. In a preferred embodiment, a silicon prism can be used to carry an IR signal, and a silicon-electrolyte interface can be electro-modulated or IR light can be photo-modulated to be used in FTIR spectroscopy with sensitivity of about 0.1 monolayer. Standard wireless communication and computing capabilities can be also integrated to perform storage, treatment/processing and transfer/receiving data/information.

Embodiments of the invention can include nano-electrodes functionalized with chemical group(s) (such as streptavidin) to provide chemical bonding to an analyte. For example, the functionalizing chemical groups can include NH₂, COOH groups, thiol chemistries, etc.

The nano-electrodes can be made of noble metals (Au, Ag, Pd, Pt, Ru, Rh, fIr, Os) or carbon (e.g., multi-wall carbon nanotubes, single-wall carbon nanotubes, graphite, diamond, etc.). Selective functionalization of electrodes can be performed by using selective deposition of chemicals through nano delivery channels. Embodiments of the invention can include analytic instrumentation coupled to the sensor array that can perform impedance spectroscopy, rest potential measurements, voltammetry, amperometry and/or conductometry to improve sensor selectivity and reduce interference.

For antibody sensor arrays, embodiments of the invention will typically need to utilize larger spots in order for reactions to go to completion in low concentration situations. In this case spot sizes can typically range from approximately 10 microns to approximately 2000 microns, preferably from approximately 100 microns to approximately 200 microns. Depending on the size and adsorption mechanism of the target analyte and the concentration, the spot size can go lower, (e.g., less than approximately 100 nm). In other cases, the spot size will need to stay large.

While not being limited to any particular performance indicator or diagnostic identifier, preferred embodiments of the sensor array can be identified one at a time by testing for the presence of sensing with respect to a known concentration of target analyte. The test for the presence of sensing can be carried out without undue experimentation by the use of a simple and conventional impedance spectroscopy experiment. Among the other ways in which to seek embodiments having the attribute of sensing guidance toward the next preferred embodiment can be based on the presence of a characteristic IR spectroscopy signal.

Embodiments of the sensor array can be identified by scanning electron microscope (SEM) cross-sections. Embodiments of the sensor array can also be identified by material analysis of devices containing sensors using techniques such as Auger spectroscopy and/or dynamic secondary ion mass spectroscopy.

Embodiments of the invention can include the use of cyclic voltammetry to characterize polarization of the electrode(s) which is affected by adsorbed organics and inorganics. Embodiments of the invention can include the use of total internal reflection IR spectroscopy to identify adsorbed organic and inorganic species. Embodiments of the invention can include integrating impedance measurement circuitry into an array and using memory array technologies to perform the electrical readout. Embodiments of the invention can include combining charge-based detection with electrical impedance spectroscopy. Embodiments of the invention can include integration of charge-based detection with electrochemical detection. Embodiments of the invention can include integration of electrical impedance spectroscopy with electrochemical detection. Embodiments of the invention can include integration of electrical readout (e.g., impedance spectroscopy, electrochemical detection and/or charge detection) to form a dense read/write array.

Nano-electrodes can be used to measure the concentration of analytes. Nano-electrodes made as inert conductors will act similar to a film/bulk electrodes while not being sensitive to the flow. Due to their nano-size, the resolution of nano-electrodes can be down to molecular/functional group level. Information about chemical species/functional groups can be stored between two electrodes with narrow spacing (e.g., from approximately 5 nm to approximately 1000 nm) by applying bias to electrodes and adsorbing/immobilizing the chemical species. These functional groups will change the structure of double electrical layer, which can then be measured by using impedance spectroscopy or other electrochemical techniques.
σ^{I}+σ^{d}=−σ^{e}
where σⁱ is the adsorbed charge; σ^{d} is the diffusion layer charge, σ^{e} is the electrode charge.
I=E/Rs Exp(−t/RsCd);
the impedance is measured as a function of frequency of the AC source.
E=I×Z; Z(W)=Zreal−jZimagine; where Zreal=R; Zimagine=1/WC

The concentration of the chemicals can also be measured (for example in the case of potentiometry) based on the Nernst equation:
E=E^{o}+(RT/zF)ln aₘ=E^{o}+(0.059/z)log aₘ.

Embodiments of the invention can include the use of nano-electrodes in combination with ion selective membranes to improve sensitivity. Embodiments of the invention can include the use of Infrared light to collect additional information about functional groups by analyzing IR vibration modes.

Embodiments of the invention can include the use of solid state electrodes fabricated to have structures and charge distributions similar to target analyte chemicals. This structure/distribution approach can be based on DNA molecular recognition ability. DNA is composed of building blocks termed nucleotides—adenine A, thymine T, guanine G and cytosine C with phosphate and sugars of adjacent nucleotides linked to form a long polymer. Nucleotides are linked in series—from one phosphate to the next sugar, to the next phosphate and so on. Information is coded into the nucleotide's sequence (order). DNA serves as a template for recreating DNA because of the obligatory pairing of A to T and G to C through NH to N and NH₂ to O bonds.

Embodiments of the invention can include a sensing mechanism based on a change in the rate of electrolysis. Self-assembled interlayer is used and its coverage can be modulated by addition of analyzed species (similar to Cu plating in solution with additives after a self-assembled monolayer of PEG/CI is formed on the surface and its surface coverage is changed when ASUPP-SPS is added to the solution and partially replaced PEG on the surface). Polarization of electrodes during electrolysis will be changed if molecular adsorbed on the electrode. This change in polarization will depend on molecular dipole, charge and functional group and can serve as molecular recognition tool. Free energy of adsorption (for organic species) is proportional to the differences in both polarizabilities and permanent dipole moments between polar species and the solvent.

The small size and space between nano-electrodes approaching molecular size can provide resolution to identify and measure individual functional groups presence. The presence of these groups can be detected, for example by measuring changing capacitance and resistivity of the double electrical layer.

Embodiments of the invention can include surface atoms on an electrode/electrolyte interface rearranging to form self-assembled surface structures consisting of regular stripes and dots depending on applied voltage and absorbate-induced mobility change with wavelengths of about 25 angstroms. Such stripe/dot patterns are also observed at the larger scale, when surfactant self-assemble at the electrode interfaces. These aggregates form micelle cylinders, hemicells, and other pattern of about 10 nm long as reported by S. Manne, and H. E. Gaub, Science 270, 1480, 1995. Self-assembled organics molecules can also produces surface structures of 50-150 nm wavelength (pitch) and about 10 nm size as reported by V. Yuzhakov, P. Takhistov, A. Miller, H-C Chang, Chaos 9, N1, 62-77, 1999.

Embodiments of the invention can include impedance spectroscopy, amperommetry, voltammetry and other electrochemical techniques used to generate a response from adsorbed analyte through the electrodes/probes. Embodiments of the invention can include the use of optical techniques such as FTIR spectroscopy can be used to identify the functional groups of analyzed chemicals species. Embodiments of the invention can recognize molecules through the use of functional groups by changing the structure of the double electrical layer at the electrode tip(s) in response to electrical, IR or other actinic signal. Embodiments of the invention can recognize molecules through the use of molecular weight by estimating diffusion coefficient. Embodiments of the invention can recognize molecules through the use of size by controlling pore size in a membrane or other kind of filter. Embodiments of the invention can recognize molecules through the use of charge (+/−) by controlling the applied potential(s). Embodiments of the invention can recognize molecules through the use of charge distribution pattern, which represents molecular structure by measuring potential/current on individually controlled nano-electrodes. Embodiments of the invention can recognize molecules through the use of electrical impedance spectrum (at one or a wide variety (sweep) of excitation frequencies). Embodiments of the invention can recognize molecules through the use of hybridization if nano-electrodes are functionalized with probes such as RNA. Embodiments of the invention can recognize molecules through the use of antibody/antigen binding if nanoprobes are functionalized with antibodies to form an antibody array or with proteins to form a protein array. Embodiments of the invention can recognize molecules through the use of peptide/protein binding using peptide aptamers. Embodiments of the invention can recognize molecules through the use of RNA aptamer/protein (or peptide) binding. Embodiments of the invention can recognize molecules through the use of redox potential.

Specific embodiments of the invention will now be further described by the following, nonlimiting examples which will serve to illustrate in some detail various features. The following examples are included to facilitate an understanding of ways in which embodiments of the invention may be practiced. It should be appreciated that the examples which follow represent embodiments discovered to function well in the practice of embodiments of the invention, and thus can be considered to constitute preferred modes for the practice of embodiments of the invention. However, it should be appreciated that many changes can be made in the exemplary embodiments which are disclosed while still obtaining like or similar result without departing from the spirit and scope of embodiments of the invention. Accordingly, the examples should not be construed as limiting the scope of embodiments of the invention.

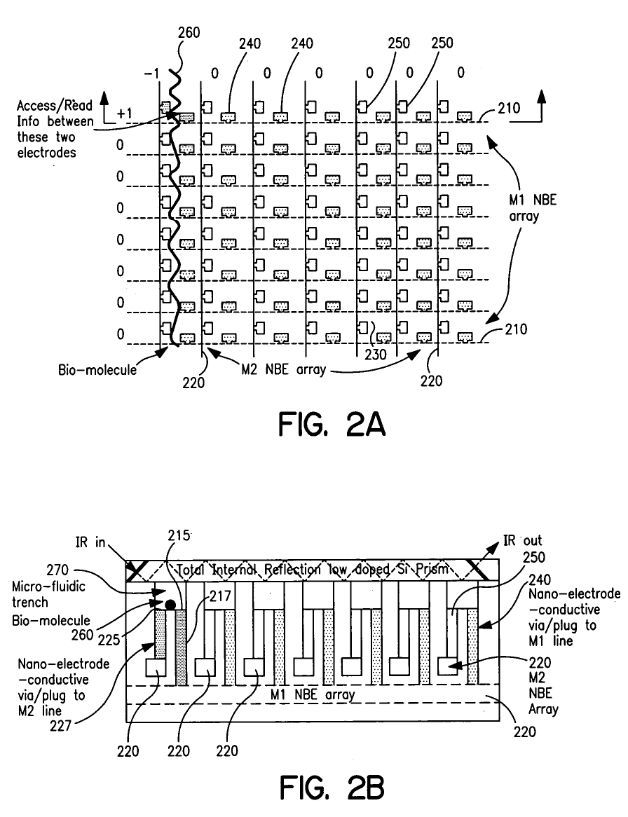

Referring to FIGS. 2A and 2B, a nano-electrode array designed as a high density memory cell array (nano-bioelectrochemical array-NBE array) is depicted. The NBE array can be based upon, and formed on top of, a CMOS (complimentary metal oxide silicon) chip. This example configures both the M1 NBE array and the M2 NBE array to one side of the sample space (i.e., the microfluidics trenches).

FIG. 2A depicts a top-view of the NBE high density memory array. It is important to note that an immobilized bio-molecule is depicted on a biased electrode with selected single bit.

FIG. 2B depicts a cross-sectional view of the NBE array. It is important to note the microfluidics trenches and a bonded silicon prism for IR total internal reflection of an IR spectroscopy signal. The NBE array can also include one or more porous membranes (not shown) upstream of the illustrated microfluidics trenches to filter the material that is fed to the trenches.

Referring to FIGS. 2A and 2B, the NBE array includes an M1 NBE array including a first plurality address lines including substantially parallel traces 210 (depicted with dashed lines). The NBE array also includes an M2 NBE array including a second plurality of address lines including substantially parallel traces 220 (depicted with solid lines). In this embodiment, traces 220 are substantially perpendicular to traces 210, thereby defining a 2 dimensional array of cells 230. Each of the cells 230 includes an M1 electrode 240 and an M2 electrode 250. A bio-molecule 260 is depicted between two M2 trace 220 of a single column of cells 230.

Referring to FIG. 2B, it can be appreciated that the bio-molecule 260 is located in one of a plurality of micro-fluidic trenches 270. A first electrode tip 215 is electronically coupled to the trace (conductive line) 210 via a conductive via/plug 217. A second electrode tip 225 is electronically coupled to one of the traces (conductive lines) 220 via a conductive via/plug 227. A total internal reflection (TIR) prism 280 is coupled to the micro-fluidic trench 270.

Optionally, one, some or all of the vias/plugs can include a transistor configured with its source and drain connected in series with that via/plug and its gate connected to the corresponding other via/plug for that cell. In this way, only the electrode tips of a row and column addressed cell would be biased (as opposed to every cell in an addressed row or addressed column having an electrode tip at the same bias state). This could provide advantages with respect to binding and/or reading with one cell without regard to basis states in other cells, especially the four nearest neighbor cells. Further, given two equal gate thresholds in a single cell of interest, by applying different row and column addressing voltages to that single cell where either the row voltage or the column voltage was above the corresponding gate threshold, but not both, the other electrode tip in that single cell would be biased with the sub-threshold voltage (as opposed to both electrode tips being biased). This could provide advantages with respect to functionalizing one of the electrode tips in a single cell without regard to simultaneously processing the other electrode in that single cell.

The operation of the embodiment illustrated in FIGS. 2A-2B will now be described. Writing, accessing and reading a single bit of information corresponding to specific functional group information regarding an adsorbed chemical species can be done between two nano-electrodes. To write information into the NBE array, a program can apply a bias to metal trace line (M1 and/or M2) to adsorb chemical species on the corresponding electrode(s) in micro-fluidics channels. To access the information, the program can apply different potentials to the row and column corresponding to the cell to be accessed (i.e. the labeled voltages +1 and −1), thereby applying a field between the two electrodes, in this instance the two darkest shaded electrodes in the upper left hand corner of the array. To read information, the program can modulate the potential applied to specific row and column to measure current and/or impedance (again in this instance, between two darkest shaded electrodes. Optionally, the program can increase the single to noise ratio by reading many times the same info in single bit (accumulate signal).

Methods of manufacturing the embodiment illustrated in FIGS. 2A-2B will now be described. Sensor array integrated circuits (cross-sectional view is shown in FIG. 2b and top-view in FIG. 2a) can be fabricated by using the following main steps. The substrate can be fabricated using standard and readily commercially available CMOS (bi-polar) chip containing semiconductor devices and metallization to store and optionally amplify and/or transfer/receive signals from the electrodes and light probes. The array of rows (M1 lines) can be fabricated by standard and readily commercially available operations of lithography, etching, metal deposition and conformal metal patterning (CMP) on top of the substrate. A first ILD (isolating layer dielectric) is deposited to isolate the array of rows from the columns. The ILD can include silica, silicon nitride and/or any other suitable insulating material. The array of columns (M2 lines) array can be fabricated by standard and readily commercially available operations of lithography, etching, metal deposition and CMP. The array of columns array can include aluminum, copper and/or any other suitable conductive material. A second ILD layer is deposited to form the electrodes and the micro-fluidics trenches.

The vias/plugs (to be used as nano-electrodes after the vias are filled with conductive material(s)) to the M1 and M2 lines, and the trenches on top of vias (micro-fluidics channels) can be fabricated using dual damascene patterning with selective via fill or using single damascene techniques with blanket conductive materials to fill the vias followed by CMP.

Damascene patterning (e.g., of copper IC interconnects) can include using photolithography and reactive plasma etching to pattern trenches, ion sputtering and electroplating to deposit metal, and CMP for removal of excess metal. A general damascene flow can begin after several layers of conventional (e.g., tungsten) contact/via plugs and (e.g., cladded aluminum) interconnects have been fabricated. A damascene patterning technique can include depositing damascene insulator films using silicon oxide or low-k dielectrics and a thin etch stop (e.g. silicon nitride and/or SiCH moities) with deep UV photolithography and plasma etch, separately defining the via and metal line trenches (i.e., “dual” patterns); completely etching vias to an underlying metal layer while lines stop partway in the dielectric, usually with the aid of an etch stop layer; after etch cleaning, depositing a copper diffusion barrier (e.g. Ta, TaN or TiN) and a thin copper “seed” film using (e.g., ion-assisted) physical vapor deposition or chemical vapor deposition (i.e., PVD or CVD); electroplating copper to overfill vias and trenches as well as the field areas; polishing (e.g., CMP) to remove the field area of copper and barrier films but stopping with the trench and via features still full of metal; depositing a capping diffusion insulator barrier (e.g., silicon nitride) by CVD. The foregoing process can be repeated for the required number of interconnect layers followed by final passivation and testing. Depositing a copper diffusion barrier (e.g., tantalum nitride) followed by depositing copper itself and provides a “seed” layer suitable for subsequent electroplate filling.

The optical probing (e.g., FTIR) total internal reflection prism can be bonded to the sensor array integrated circuit. Alternatively, the prism can be silica based instead of silicon based, and be fabricated on top of the sensor array integrated circuit in-situ using readily commercially available sol-gel techniques. Optionally, chemical selective membranes and/or films can be fabricated and/or attached to the sensor array integrated circuit. The sensor array integrated circuits can be packaged in a hand held device. In addition, the hand held device can include micro valve, piping, pumps, RF, display, sample port capabilities, etc. An additional operation can include selective functionalization of electrodes in-situ (i.e., in the microfluidics channels).

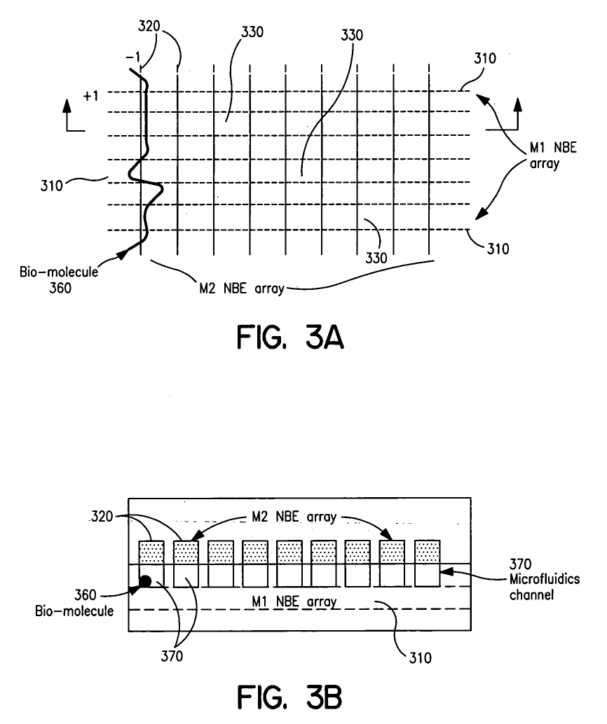

Referring to FIGS. 3A and 3B, another embodiment of a nano-electrode (conductive lines) array configured as a high density memory cell array (nano-bioelectrochemical array-NBE array) is depicted. Again, the NBE array can be based upon, and formed on top of, a CMOS (complimentary metal oxide silicon) chip. This example configures the M1 NBE array on a first side of the sample space(s) (i.e., the microfluidics trenches) and the M2 NBE array on a second side of the sample space.

FIG. 3A depicts a top-view of the nano-electrode array. Again, it is important to note that an immobilized bio-molecule is depicted on a biased electrode with selected single bit.

FIG. 3B depicts a cross-sectional view of the nano-electrode (nano-tube) array. It is important to note the microfluidics trenches. The NBE array can also include one or more porous membranes (not shown) upstream of the illustrated microfluidics trenches to filter the material that is fed to the trenches.

Referring to FIGS. 3A and 3B, the NBE array include an M1 NBE array including a first plurality address lines including substantially parallel traces 310 (depicted with dashed lines). The NBE array also include an M2 NBE array including a second plurality of address lines including substantially parallel traces 320 (depicted with solid lines). In this embodiment, traces 310 are substantially perpendicular to traces 310, thereby defining a 2 dimensional array of cells 330. A bio-molecule 360 is depicted between two M2 electrodes 320 of a single column of cells 330.

Referring to FIG. 3B, it can be appreciated that the bio-molecule 360 is located (at least in-part) in one of a plurality of micro-fluidic trenches 370. A significant advantage of this embodiment is that the traces (conductive lines) 310, 320 themselves function as the electrode tips, thereby obviating the need for tip structures and/or via/plug structures.

The operation of the embodiment illustrated in FIGS. 3A and 3B will now be described. Information in this type of array can be written by using a program to apply a potential to conductive lines in row and/or column causing adsorption of chemical species on conductive line surface exposed in micro-fluidic channel/trench. The information can be accessed and/or read on the intersection points of conductive lines in micro-fluidic channel by the program as in the previous example.

Methods for manufacturing the embodiment illustrated in FIGS. 3A-3B will now be described.

The substrate can be fabricated with standard and readily commercially available CMOS (bi-polar) chip techniques to provide semiconductor devices and metallization to amplify, treat, store and transfer/receive signals from the electrodes and/or light probes. The first array of rows (array M1 for nano-electrode arrays) can be fabricated by standard and readily commercially available operations of lithography, etching, metal deposition and CMP. A first ILD layer can be deposited to isolate column and arrow and form sacrificial material (e.g., thermally decomposable polymer such as Unity or selectively etchable material such as carbon) in trenches etched in the first ILD. A second ILD layer can be similarly deposited followed by fabrication of the second array of columns (array M2 for nano-electrode arrays) by standard and readily commercial available operations of lithography, etching, metal deposition and CMP. The micro-fluidics channels can be fabricated between columns and rows by removing the sacrificial material(s).

A practical application of embodiments of the invention that have value within the technological arts is integrating chemical and/or biological sensing with computing and communication. There are virtually innumerable uses for embodiments of the invention, all of which need not be detailed here.

Embodiments of the invention can be cost effective and advantageous for at least the following reasons. In general, embodiments of the invention improve quality and/or reduce costs compared to previous approaches.

A technical advantage that can be provided by an embodiment of the invention includes increased functionality and performance of sensors by enabling molecular recognitions with solid sensors electrodes (5-1000 nm size and space between electrodes, down to 0.8 nm size electrode if SW CNT is used) manufactured by mature semiconductor technology and designed as memory cell arrays which are capable of writing information on (from) bio- and/or chemical-molecules by immobilizing (e.g., adsorption) chemical species on the biased electrodes as well as accessing and reading single bits of information corresponding to specific chemical functional groups contained between two nano-electrodes by measuring current between these electrodes having unique combination of applied alternative (pulse) voltage. Reading of information can be repeated (accumulated) and a spectroscopic signal can be modulated (electromodulated or photo-modulated) to increase single to noise ratios.

Another technical advantage that can be provided by an embodiment of the invention includes reducing cost by integrating thousands (even millions) of sensors on a substrate using semiconductor technology processing/operations.

Another technical advantage that can be provided by an embodiment of the invention includes performing continuous point of care analysis and diagnosis, thereby responding to and containing hazard/health issues while reducing risk of complications. For example, the monitoring of blood potassium levels can give early warning of the steady increase that often precedes an embolism, providing sufficient time for clinical countermeasures. Similarly, immobilization of a multiplicity of affinity reagents such as antibodies or aptamers, each preferentially selective to a different set of proteins can provide ability for the array to detect compound patterns indicative of early onset of disease, drug toxicity detection, treatment selection, disease diagnosis/prognosis, and tissue typing.

Another technical advantage that can be provided by an embodiment of the invention includes increasing sensor reliability by using mature semiconductor technology to fabricate and integrate sensors.

Another technical advantage that can be provided by an embodiment of the invention includes analysis of chemical species performed by changing the rate of electrolysis on nano-electrodes when organics species are added to the solution and adsorbed on the electrode surface. For example in Cu plating, the addition of ethers types of additives suppressed the deposition rate while addition of anti-suppressor (such as SPS) increases the deposition rate. Nano-electrodes are not sensitive to flow since the rate of electrolysis equivalent to rate of diffusion.

Another technical advantage that can be provided by an embodiment of the invention includes analysis of chemical species performed by measuring the electrical impedance between electrodes, in which said electrical impedance will change when a captured affinity reagent that has been ‘written’ into the array encounters its target analyte.

Another technical advantage that can be provided by an embodiment of the invention includes analysis of chemical species performed by measuring the charge (electric field) present in the vicinity of the electrode, for instance through the use of an ion-sensitive transistor where the electrode acts as the gate of the transistor. Materials such as single stranded DNA may be used as the affinity reagent, with hybridization used as the target capture mechanism. The charge on the DNA then modifies the conductivity properties of the transistor, which is in turn detected using a sense amplifier comparable to those used to detect charge stored in traditional dynamic memory cells.

Another technical advantage that can be provided by an embodiment of the invention includes analysis of biological species performed by building an artificial anti-body using nano-electrodes. Solid-state electrodes are manufactured in pattern/shape and charge distribution similar to analytes/molecular to enable molecular recognition ability.

The terms a or an, as used herein, are defined as one or more than one. The term plurality, as used herein, is defined as two or more than two. The term another, as used herein, is defined as at least a second or more. The terms “comprising” (comprises, comprised), “including” (includes, included) and/or “having” (has, had), as used herein, are defined as open language (i.e., requiring what is thereafter recited, but open for the inclusion of unspecified procedure(s), structure(s) and/or ingredient(s) even in major amounts. The terms “consisting” (consists, consisted) and/or “composing” (composes, composed), as used herein, close the recited method, apparatus or composition to the inclusion of procedures, structure(s) and/or ingredient(s) other than those recited except for ancillaries, adjuncts and/or impurities ordinarily associated therewith. The recital of the term “essentially” along with the terms “consisting” or “composing” renders the recited method, apparatus and/or composition open only for the inclusion of unspecified procedure(s), structure(s) and/or ingredient(s) which do not materially affect the basic novel characteristics of the composition. The term coupled, as used herein, is defined as connected, although not necessarily directly, and not necessarily mechanically. The term any, as used herein, is defined as all applicable members of a set or at least a subset of all applicable members of the set. The term approximately, as used herein, is defined as at least close to a given value (e.g., preferably within 10% of, more preferably within 1% of, and most preferably within 0.1% of). The term substantially, as used herein, is defined as largely but not necessarily wholly that which is specified. The term generally, as used herein, is defined as at least approaching a given state. The term deploying, as used herein, is defined as designing, building, shipping, installing and/or operating. The term means, as used herein, is defined as hardware, firmware and/or software for achieving a result. The term program or phrase computer program, as used herein, is defined as a sequence of instructions designed for execution on a computer system. A program, or computer program, may include a subroutine, a function, a procedure, an object method, an object implementation, an executable application, an applet, a servlet, a source code, an object code, a shared library/dynamic load library and/or other sequence of instructions designed for execution on a computer or computer system.

All the disclosed embodiments of the invention disclosed herein can be made and used without undue experimentation in light of the disclosure. Embodiments of the invention are not limited by theoretical statements recited herein. Although the best mode of carrying out embodiments of the invention contemplated by the inventor(s) is disclosed, practice of the embodiments of the invention is not limited thereto. Accordingly, it will be appreciated by those skilled in the art that the embodiments of the invention may be practiced otherwise than as specifically described herein.

It will be manifest that various substitutions, modifications, additions and/or rearrangements of the features of the embodiments of the invention may be made without deviating from the spirit and/or scope of the underlying inventive concept. It is deemed that the spirit and/or scope of the underlying inventive concept as defined by the appended claims and their equivalents cover all such substitutions, modifications, additions and/or rearrangements.

All the disclosed elements and features of each disclosed embodiment can be combined with, or substituted for, the disclosed elements and features of every other disclosed embodiment except where such elements or features are mutually exclusive. Variation may be made in the steps or in the sequence of steps defining methods described herein.

Although the sensor array described herein can be a separate module, it will be manifest that the sensor array(s) may be integrated into the system with which it is (they are) associated. Similarly, although the hand held device described herein can be a separate module, it will be manifest that the hand held device(s) may be integrated into the system with which it is (they are) associated.

The individual components need not be formed in the disclosed shapes, or combined in the disclosed configurations, but could be provided in all shapes, and/or combined in all configurations. The individual components need not be fabricated from the disclosed materials, but could be fabricated from all suitable materials. Homologous replacements may be substituted for the substances described herein. Agents that are both chemically and physiologically related may be substituted for the agents described herein where the same or similar results would be achieved.

The appended claims are not to be interpreted as including means-plus-function limitations, unless such a limitation is explicitly recited in a given claim using the phrase(s) “means for” and/or “step for.” Subgeneric embodiments of the invention are delineated by the appended independent claims and their equivalents. Specific embodiments of the invention are differentiated by the appended dependent claims and their equivalents.

1. An apparatus, comprising:
a condensed array addressed device including a plurality of addressable cells, each of the plurality of addressable cells including at least two electrodes; and
a spectroscope optically coupled to the condensed array addressed device.

2. The apparatus of claim 1, wherein the spectroscope includes an infrared spectroscope.

3. The apparatus of claim 2, wherein the infrared spectroscope includes a Fourier transform infrared spectroscope.

4. The apparatus of claim 2, wherein an infrared spectroscope signal from the infrared spectroscope is electromodulated by applying potential between the at least two electrodes in at least one of the plurality of cells.

5. The apparatus of claim 2, wherein an infrared spectroscope signal from the infrared spectroscope is photo-modulated by applying a modulated UV-VIS signal to a surface of at least one of the at least two electrodes.

6. The apparatus of claim 1, wherein the condensed array addressed device includes a waveguide total internal reflection prism optically coupled to a region proximal electrodes of a cell and the spectroscope is optically coupled to the waveguide.

7. The apparatus of claim 6, wherein the waveguide includes a total internal reflection prism and the spectroscope is optically coupled to the total internal reflection prism.

8. The apparatus of claim 1, wherein each of the plurality of addressable cells includes an individually addressable cell.

9. The apparatus of claim 8, wherein the individual addressable cell includes a first individually addressable electrode and a second individually addressable electrode.

10. The apparatus of claim 1, wherein each of the plurality of addressable cells includes a pair of electrodes that are less than approximately 200 microns in size and the spacing of the electrodes is less than approximately 200 microns.

11. The apparatus of claim 10, wherein each of the pair of electrodes are less than approximately 100 nm in size.

12. The apparatus of claim 10, wherein the spacing of the pair of electrodes is less than approximately 100 nm.

13. The apparatus of claim 10, wherein each of the pair of electrodes includes at least one member selected from the group consisting of single-walled carbon nanotubes and silicon nano-wires.

14. The apparatus of claim 1, wherein the plurality of addressable cells define a plurality of sensor elements configured as an array, wherein each of the sensor elements is functionalized to interact with one or more target molecules; and further comprising control circuitry coupled to the sensor elements, wherein the control circuitry is configured to detect interactions of the sensors with the target molecules.

15. The apparatus of claim 14, wherein the plurality of sensor elements are configured as a two-dimensional array and are addressable using memory cell techniques.

16. The apparatus of claim 15, wherein the plurality of sensor elements are addressable by corresponding rows and columns of the two-dimensional array.

17. The apparatus of claim 14, wherein the plurality of sensor elements are configured as a high-density array.

18. The apparatus of claim 14, further comprising memory coupled to the control circuitry, wherein the control circuitry is configured to store data corresponding to the plurality of sensor elements in the memory.

19. The apparatus of claim 1, further comprising a microfluidic channel coupled to at least one of the addressable cells.

20. The apparatus of claim 1, further comprising a selective membrane coupled to at least one of the addressable cells.

21. The apparatus of claim 20, wherein the selective membrane includes at least one member selected form the group consisting of chemically selective membranes and biologically selective membranes.

22. A method comprising:
providing a spectroscope optically coupled to an integrated array of cells, each of the cells including a sensor element; and
functionalizing each of the sensor elements to interact with a target molecule.

23. The method of claim 22, further comprising exposing each of the sensor elements to a sample and detecting whether the target molecule in the sample interacts with each of the sensor elements.

24. The method of claim 23, wherein detecting includes measuring an optical property.

25. The method of claim 24, wherein measuring includes infrared spectroscopy.

26. The method of claim 25, wherein infrared spectroscopy includes Fourier transform infrared spectroscopy.

27. The method of claim 23, wherein measuring includes conveying an optical signal via total internal reflection.

28. The method of claim 23, wherein detecting further includes measuring an electrical property.

29. The method of claim 28, wherein measuring includes impedance spectroscopy.

30. The method of claim 28, wherein measuring the electrical property includes individually addressing one of the cells.

31. The method of claim 30, wherein individually addressing one of the cells includes individually addressing one of the sensor elements and measuring the electrical property independent of any of the other sensor elements.

32. The method of claim 30, further comprising repeating measuring the electrical property and integrating to reduce a signal to noise ratio associated with the integration.

33. A method, comprising: determining whether a target molecule has coupled to a condensed array addressed device by characterizing a subsequent rate of electrolysis on the condensed array addressed device.

34. The method of claim 33, wherein coupled includes chemical bonding.

35. The method of claim 33, wherein characterizing includes measuring the polarization of an electrode during electrolysis.

36. A data structure comprising results obtained using the method of claim 33.

37. A method, comprising:
fabricating a condensed array addressed device including forming vias to connect an electrodes to an address line and filling the via with conductive material to define a plug including damascene patterning at least one member selected from the group consisting of the via, the plug and the address line.

38. The method of claim 37, wherein the via is etched to the address line and another structure is simultaneously etched to a stop feature.

39. The method of claim 37, wherein damascene patterning includes dual damascene patterning including separately defining the via and address line.

40. A condensed array addressed device produced by the method of claim 37.
